(19) United States
(12) Patent Application Publication
(10) Pub. No.: US 2004/0115771 A1
(43) Pub. Date: Jun. 17, 2004

(54) Recombinant collagen-like proteins

(75) Inventor: Andrzej Fertala, Voorhees, NJ (US)

Correspondence Address: David S. Resnick, Esq., NIXON PEABODY LLP, 101 Federal Street, Boston, MA, 02110, US

(73) Assignee: Thomas Jefferson University, Philadelphia, PA (US)

(21) Appl. No.: 10/671,005

(22) Filed: Sep. 25, 2003

Related U.S. Application Data
(60) Provisional application No. 60/414,143, filed on Sep. 27, 2002.

(51) Int. Cl. C07K 14/78; C07H 21/04

(52) U.S. Cl. 435/69.1; 435/320.1; 435/325; 530/356; 435/252.3; 536/23.5

(57) ABSTRACT

The present invention relates generally to collagen proteins, and particularly to recombinant collagen-like proteins consisting of multiple homogeneous domains with high density of biologically active sites.

### CROSS-REFERENCE

This application claims the benefit of U.S. Provisional Application No. 60/414,143, filed Sep. 27, 2003 under U.S.C. § 1119(e).

### GOVERNMENT FUNDING

This invention was made with government support under NAG9-1342 awarded by the National Aeronautics and Space Association. The government has certain rights in the invention.

### FIELD OF THE INVENTION

The present invention relates generally to collagen proteins, and particularly to recombinant collagen-like proteins consisting of multiple homogeneous domains with high density of biologically active sites.

### BACKGROUND OF THE INVENTION

Collagen is one of the most attractive materials for a scaffold for tissue repair. In addition to its mechanical and structural characteristics, it is notable that the scaffold promotes cell attachment and migration and allows preservation of a specific phenotype. Moreover, a scaffold is an important device for delivery into a site of injury growth factors that promote tissue repair. The ability to engineer modified collagen-like molecules with novel structural and biological characteristics to produce materials for cartilage repair and tissue engineering would be beneficial.

Cartilage is an important target in tissue engineering. Millions of individuals are incapacitated by the destruction of articular cartilage by trauma or disease processes such as osteoarthritis or rheumatoid arthritis. However, the tissue does not repair itself. A greater understanding of the mechanism of attachment and migration of chondrocytes through collagen matrices designed to promote cartilage repair and interaction of collagen with bone morphogenetic proteins is required.

Collagen II is the most abundant protein of cartilage, and it forms a network of fibrils that are extended by proteoglycans and, thereby, provide the resistance of cartilage to pressure. One approach to tissue engineering of cartilage has been to isolate chondrocytes from biopsy specimens of normal cartilage, expand the chondrocytes in culture, and then use the chondrocytes to re-surface degenerated articular cartilage in the same patient ¹⁻³. A related strategy is to use chondrocyte precursors from bone marrow^{4, 5} or periosteum ⁶.

In cartilage, chondrocytes are embedded in a matrix of collagen fibrils and proteoglycans¹⁰. Over six different types of collagen have been identified in cartilage¹¹, but collagen II accounts for 95% of the total collagen¹². The role of collagen II in the organization of cartilage was demonstrated in mice with an inactivated COL2A1 gene¹³. Cartilage of homozygous animals consisted of highly disorganized chondrocytes and, as demonstrated by Yang et al.¹⁴, the cells underwent a rapid apoptosis.

A large number of materials have been tested for use in cartilage repair. These include synthetic biodegradable, non-biodegradable polymers, hydrogels^{7, 8} and collagen purified from animal sources⁹. The advantage of synthetic polymers is that they make it possible to control physical properties such as texture, porosity, density and mechanical strength. However, most synthetic materials do not have optimal biological characteristics. Thus, there still exists a need for novel approaches to cartilage repair and tissue engineering.

### DESCRIPTION OF THE INVENTION

Before the present proteins and methods are described, it is understood that this invention is not limited to the particular methodology, protocols, cell lines, vectors, and reagents described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a host cell” includes a plurality of such host cells.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the cell lines, vectors, and methodologies which are reported in the publications which might be used in connection with the invention. Nothing herein is to be construed as an admission that the invention is not entitled to antedate such disclosure by virtue of prior invention.

The present invention provides recombinant collagen-like proteins containing multiple domains of collagen II region, e.g., D1, D2, D3, D4 or D5. In one embodiment, the recombinant proteins of the present invention possess a higher density of biologically active sites available for specific interactions and have superior properties critical for successful tissue engineering, such as for preparation of a scaffold to form cartilage in vitro. For example, the recombinant proteins of the present invention possess regions with high density of sites critical for attachment and migration of chondrocytes as well as for binding of bone morphogenic protein 2 (BMP-2).

In one embodiment, the present invention provides a recombinant collagen-like protein comprising a multi-domain of the formula (D-D)x, wherein D is selected from the D1, D2, D3, D4 or D5 protein domain of collagen and each D is identical, and wherein x is 1-5. In a preferred embodiment, x is 2. Preferably, x is a number that results in a protein that retains essentially the same structure (e.g., length, triple helical conformation) as the native protein. The collagen-like protein of the present invention may also include other domains such as those set forth in Table 1 below. A preferred protein has the structure CtD5D4D4D4D1Nt.

The present invention also provides a nucleic acid sequence encoding the recombinant collagen-like protein. The nucleic acid may be DNA, cDNA or RNA. The nucleic acid may be part of a host cell.

A DNA cassette system may be utilized to engineer constructs that encode collagen-like proteins consisting of multiple homogeneous domains of human collagen II, e.g., domains D1, D2, D3, D4 or D5 of the triple helix. The method disclosed in detail by Arnold et al.,²⁹ may be used. A preferred domain is the D4 region of the triple helix of human collagen II, which is critical for integrin-mediated interaction between chondrocytes and collagen II.

The collagen-like protein can be produced in a recombinant DNA system, for example, as described in U.S. Pat. Nos. 5,405,757 and 5,593,859, and incorporated herein by reference in their entirety. cDNA cassettes can be synthesized as described in detail by Arnold et al.,²⁹ to produce the recombinant collagen-like protein. DNA constructs can be expressed in HT-1080 cells and recombinant proteins can be purified from cell culture media as described by Fertala et al.³⁰. An example of how to make a recombinant collagen-like proteins of the present invention is shown infra.

The purified protein can be used in cartilage repair and tissue engineered constructs.

The invention will be further characterized by the following examples which are intended to be exemplary of the invention.

### EXAMPLE 1

Methods and Materials

Procollagen II DNA cassette system—To produce genetically engineered collagen II variants lacking consecutive fragments of 234 amino acids, defined here as D-periods because of correlation with the D-periodicity of collagen fibril³⁴, cDNA cassettes were synthesized as described in detail by Arnold et al.²⁹ DNA constructs were expressed in HT-1080 cells and recombinant procollagens were purified from cell culture media as described by Fertala et al.³⁰.

Human chondrocytes—Human chondrocytes were isolated from fetal epiphyseal cartilage removed under sterile conditions from femoral heads, knee condyles and tibial plateaus. Isolated chondrocytes were cultured in a suspension in tissue culture dishes coated with poly-HEMA (poly(2-hydroxyethyl methacrylate); Polysciences, Inc., Malvern, Pa.) according to the method described by Reginato et al.³⁵.

Preparation of the microtiter plates for the cell attachment and the spreading assays—To coat microtiter plates, collagen II samples dissolved in 0.1 M acetic acid at a concentration of 50 μg/ml were added to microtiter plates and allowed to dry under a laminar flow hood over night. The plates were then rinsed with phosphate buffered saline (PBS) and blocked with heat denatured bovine serum albumin (BSA; Sigma).

Seeding of chondrocytes on recombinant collagens II variants—Human chondrocytes were cultured in a suspension in tissue culture plates coated with poly-HEMA. To isolate chondrocytes the cell aggregates were transferred to the culture medium containing 2 mg/ml of trypsin and 2 mg/ml of collagenase. After 2 h of incubation, released chondrocytes were passed through a 70-μm nylon filter and collected in a 50 ml conical tube. The cells were sedimented by centrifugation at 1,500 rpm for 10 minutes. Subsequently, the cells were washed 5 times with DMEM supplemented with 10% fetal bovine serum, transferred to a fresh tissue culture dish coated with poly-HEMA, and incubated in a tissue culture incubator. After a period of 2 h, the cells were washed with serum-free DMEM, counted, and suspended to 2×10⁵ cells/ml in DMEM, 10% BSA. Fifty microliters of PBS containing 0.1 mg/ml of MgCl₂ and 0.1 mg/ml of CaCl₂ were added to each well of a microtiter plate, followed by 50 μl of the cell suspension. The cells were allowed to attach to the plates for 3 hours. In the experiments with inactivation of 01 integrins, anti-human β1 integrin antibodies (Life Technologies Inc.), diluted 1:100, were added to the wells prior to the addition of the cell suspension. Microtiter plates were incubated for 3 h, and the adhesion and the spreading of chondrocytes were evaluated.

Attachment of chondrocytes to the collagen II variants—After three hours of culture, the cell layer was washed with PBS containing MgCl₂ and CaCl₂ and fixed by the addition of 10 μl of a 50% (w/v) glutaraldehyde solution. After 1 h, the wells were rinsed with water, and the cells were stained with 1% solution of crystal violet in 200 mM MES (2-[N-morpholino]ethanesulfonic acid), pH 6.0, for 30 min. at room temperature. The excess dye was washed off with water and the cell-bound dye was dissolved with 100 μl of 10% (v/v) acetic acid. The absorbance was read at 570 nm. Results from five independent experiments were analyzed using the Cricket Graph statistical program (Cricket Software, Malvern, Pa.).

Spreading of chondrocytes on recombinant collagens II lacking specific D-period—To evaluate the spreading of chondrocytes seeded on collagen II with deleted D-periods after three hours of culture, the cells were fixed by an addition of 10 μμt of a 50% (w/v) glutaraldehyde solution directly to the wells and then stained with Giemsa stain (Sigma). To determine the percentage of the spread cells, the surface area of cells was measured. Morphometric analysis of cells was done with an inverted microscope (Olympus IX50, Olympus, Japan) equipped with a digital camera (Photometrics Systems) and connected to a personal computer. Surface areas of the chondrocytes from five non-overlapping areas of a single well were measured using the Phase3 Imaging program (Imaging Systems). Data from five independent experiments were collected and analyzed with the Cricket Graph program.

Synthesis of three-dimensional nanofibrous matrices containing recombinant collagen II—Nanofibrillar matrices were synthesized using polymers with free NH₂ groups for the covalent binding of collagen³⁶. In brief, poly (L-lactic acid) (Mw 200,000; Polysciences, Inc) was mixed with poly(&-CBZ-L-lysine) (Mw 260,000; Sigma) at a 4:1 ratio. The carbobenzoxy (CBZ)-protected form of L-lysine was used to prevent involvement of side chain groups in the formation of a CONH bond during peptide synthesis. A mixture of polymers was then dissolved in chloroform and used to generate nanofibrillar material in the electrostatic spinning process (FIG. 2). In this non-mechanical technique a high electric field is generated between a polymer fluid contained in a glass syringe with a capillary tip and a metallic collection screen. When the voltage reaches a critical value, the charge overcomes the surface tension of the deformed drop of the suspended polymer solution created on the capillary tip, and a jet is produced. The electrically charged jet undergoes a series of electrically induced bending instabilities during its passage to the collection screen that results in hyper-stretching of the jet. This process is accompanied by the rapid evaporation of the solvent. The dry fibers are accumulated on the surface of the collection screen, resulting in a non-woven mesh of nanofibers. Covalent binding of collagen was carried out according to the method developed by Zheng and collaborators³⁶ Briefly, to activate CBZ protected-amino groups, the matrices were placed in a 4.5 M HCl solution in glacial acetic acid and incubated for 30 min. at 37° C. The samples were neutralized by an addition of 0.1 M sodium carbonate and then stored in sterile water at 4° C. Recombinant collagen stock solutions were diluted to a final concentration of 200 μg/ml with 10 mM MOPS (3-(N-Morpholino)propanesulfonic acid), adjusted to pH 4.5, containing 5 mg/ml of water soluble carbodiimide (1-ethyl-3-[3-bimethylaminopropyl] carbodiimide; Pierce). The activated amino groups were permitted to react with collagen for 48 h at 4° C. Unbound collagen was then removed by a washing of the matrices with 10 mM HCl, followed by a washing with water. The efficiency of incorporation of collagen into nanofibrous matrices was determined by an analysis of the hydroxyproline content after acid hydrolysis and reaction with p-dimethylaminobenzaldehyde³⁸.

Growth of chondrocytes in three-dimensional nanofibrous scaffold—The nanofibrous scaffolds coated with collagen II variants were placed into separate wells of a microtiter plate. Chondrocytes were seeded onto the scaffolds at 10,000 cells/well and cultured for up to 50 days. Fifty percent of the media supplemented with 40 μg/ml of ascorbic acid was changed every 48 h. In the experiments with the blocking of β1 integrins, the monoclonal anti-human β1 integrin antibodies were added to the wells prior to the addition of the cell suspension. After 48 h of culture, the cells seeded onto nanofibrillar matrices were examined by scanning electron microscopy. In addition, after 50 days, the morphology of the synthesized matrix was examined by light microscopy, and the sub-structure of synthesized extracellular matrix was examined by transmission electron microscopy.

Analysis of secretion of collagen II and collagen IX—Proteins secreted into the media by chondrocytes cultured for 50 days in matrices coated with the full length collagen II were precipitated with polyethylene glycol (8,000 Mw; Sigma) at concentration of 5% (w/v). The proteins were then collected by centrifugation at 13,000×g for 30 min. at 4° C., dissolved in 0.1 M Tris-HCl buffer (pH 7.4) containing 0.4 M NaCl, 25 mM EDTA, and 0.04% NaN₃. Consequently, collagens II and IX were examined by SDS-polyacrylamide gel electrophoresis under reducing conditions followed by electroblotting and Western analysis with anti-collagen, type-specific antibodies (Chemicon, Inc). Recombinant collagen IX, used as a marker, was a kind gift from Dr. Leena Ala-Kokko (Tulane University, New Orleans, LO).

Results

Synthesis of recombinant collagen II—As shown previously³⁹, all recombinant variants of collagen II were triple helical at physiological temperature (FIG. 1).

Distribution of sites for binding of chondrocytes to collagen II—Human chondrocytes were seeded onto collagen variants lacking specific D periods. After 3 h of incubation, the cell layer was washed with PBS containing Ca²⁺ and Mg²⁺ ions, fixed with 5% glutaraldehyde, and stained with crystal violet. The dye was dissolved in 10% acetic acid, and the absorbance was measured at 570 nm. The number of attached chondrocytes was the same in all analyzed samples, as indicated by similar values of the absorbance (FIG. 3). These results indicate that the amino acid sequences important for binding of chondrocytes to collagen II are uniformly distributed throughout the collagen II monomer.

Spreading of chondrocytes on collagen II variants—Human fetal chondrocytes were seeded onto the microtiter plates coated with collagen II variants. Cells were allowed to interact with collagen for 3 h. Subsequently, the cells were fixed and stained with Giemsa stain and examined with a light microscope (FIG. 4). The cells grown on plates coated with full-length collagen II, -D1, -D2, and -D3 collagen II had spread morphology. In contrast, most of the cells grown on the -D4 collagen II or BSA-coated plates remained spherical. The extent of the spreading of chondrocytes was analyzed with an inverted microscope equipped with a digital camera, and was expressed as the cell surface area. Mean values of the surface areas of chondrocytes cultured on full-length collagen and collagens with deleted D1, D2 and D3 periods were in the range between 560 μm² and 670 μm². The surface area of chondrocytes grown on collagen II with deleted D4 period or on BSA was approximately 450 μm. The results were also expressed as a percentage of the cells with the surface area equal to or greater than the mean value of the surface area of the cells grown on the full-length collagen II. Full-length collagen II, -D1, -D2 and -D3 collagen II supported the spreading of about 40% of chondrocytes cultures for 3 h. In contrast, collagen II lacking the D4 period supported the spreading of only about 15% of the cells, a value similar to that obtained with chondrocytes grown on the plates coated with BSA (see FIG. 4). Therefore, although amino acid sequences for the binding of chondrocytes are uniformly distributed throughout the collagen II monomer, the sequences for spreading of the cells are located primarily in the D4 period (amino acids 704 to 938).

Role of the β1 integrin in the binding and spreading of chondrocytes on collagen II. To analyze the role of β1 integrins in collagen I-chondrocytes interaction, anti-β1 integrin antibodies were used to specifically block the β1 integrin-dependent attachment. The antibodies inhibited attachment of chondrocytes to all collagen II constructs by more than 50% (FIG. 3). In addition, anti-β1 integrin antibodies inhibited the spreading of chondrocytes on full length, -D1, -D2 and -D3 collagens to about 20%. In the samples with -D4 collagen II, spreading was reduced to about 12% (FIG. 4). These results indicate that β1 integrins mediate both binding and spreading of chondrocytes on collagen II. Although β1 integrin binding sites are uniformly distributed throughout the collagen II triple helical domain, the β1 integrin-mediated spreading of chondrocytes depends on interactions with amino acid residues located in the D4 region of collagen II.

Three-dimensional nanofibrous matrix—Three-dimensional matrices were prepared from mixtures of poly(L-lactic acid) and poly(ε-CBZ-lysine) by the electrostatic spinning method. The matrices were coated with genetically engineered recombinant collagen II variants and used as a support for chondrocyte attachment and spreading. The amount of collagen bound to the surface of the nanofibers was about 2 μg/mg, and non-specific binding of collagen II to the non-activated polymer was about 0.1 μg/mg. The average diameter of a nanofiber was 360 nm, and the average size of a single pore in the fibrous network was 2.1 μm. The thickness of an average material was 0.1 mm. The continuity of nanofibrous structures was interrupted by the presence of bead-like structures (FIG. 5) that were formed during the process of electrostatic spinning. As described by Fong et al.⁴⁰, the presence of such beaded nanofibers can be explained by the capillary breakup of the electrostatic spinning jets due to surface tension.

Growth of chondrocytes on nanofibrous matrices —To study how the different collagen II regions promote cell attachment and migration of chondrocytes through three-dimensional matrices, nanofibrous materials coated with collagen II variants were fabricated and used in the migration assays. As indicated in FIG. 5, cells seeded onto matrices coated with full-length collagen and -D3 collagen migrated into cavities of a scaffold. Cells seeded onto matrices coated with -D1 or -D2 collagen II variants (data not shown) showed similar behavior. In contrast, chondrocytes seeded onto matrices coated with -D4 collagen or bovine serum albumin formed clusters and remained on a surface of a nanofibrous scaffold (FIG. 5). As indicated in FIG. 6, the presence of anti-β1 integrin antibodies abolished the ability of chondrocytes to migrate into matrices coated with full-length collagen. Therefore, these results may support the observation (see FIG. 4) that the migration of chondrocytes on collagen II depends on the interaction of β1 integrins with amino acid sequences located in the D4 period of the collagen II molecule.

Nanofibers are attractive materials because they provide a large surface area for attachment of cells. To date it has not been established whether nanofibrillar matrices are able to support long-term cultures of chondrocytes. Hence, to ensure continuous synthesis of cartilaginous proteins, the secretion of procollagen II and collagen IX was analyzed after 50 days of culture. As demonstrated by Western blot analysis (FIG. 7), chondrocytes secreted procollagen II and collagen IX. Procollagen II was partially processed, most likely because of activity of procollagen processing enzymes. The presence of high migrating bands detected by the anti-collagen IX antibodies is probably a result of the binding of collagen IX to collagen II and partially processed procollagen II.

The morphology of synthesized matrix was examined by light microscopy and transmission electron microscopy. As determined by Alcian blue staining (not shown), there was deposition of proteoglycans in an upper layer of a scaffold. Transmission electron microscopy analysis of matrices showed that collagen fibrils were deposited in matrix cavities (FIG. 8). These fibrils had an apparent banding and were about 20 nm in diameter. Therefore, throughout a 50-day culture on nanofibrillar matrices coated with recombinant full-length collagen II, chondrocytes maintained their phenotype and formed a cartilage-like matrix. Because the cells were seeded only on one side of a scaffold, the synthesis of extracellular matrix was limited to the upper layer only. Since the chondrocytes seeded onto non-coated fibers remained on the surface of a scaffold after 48 h of culture (not shown), we did not attempt to culture these cells over a period of 50 days.

Discussion

The results presented here extend previous observations that the intercommunication between chondrocytes and the extracellular matrix involves site-specific interactions between integrins and collagen II. Previous attempts to localize the regions of collagen II critical for contact with chondrocytes lacked the ability to generate well-defined triple helical segments of collagen II that would cover the entire amino acid sequence of the monomer. In our studies, these problems were overcome by the use of genetically engineered procollagen II variants in which the amino acid sequences that correspond to the specific collagen D periods were purposely deleted.

The data suggest that attachment and spreading of cells are controlled by different mechanisms. A similar adhesion of cells to all collagen II variants indicates that the collagen II a chains of over 1000 amino acids each contain uniformly distributed sites for the attachment of chondrocytes. Since the adhesion of chondrocytes to the collagen II variants was reduced by anti β1 integrin antibodies to about 15%, a value similar to that obtained with chondrocytes grown on bovine serum albumin coated plates, the main mechanism of the attachment involves P1 integrins. It was postulated that the α1β1, α2β1 and α10β1 integrins play a key role in the interaction of chondrocytes with collagen II^{23, 41, 42}. However, the exact molecular mechanism of integrin mediated adhesion to collagen II is not known. Biochemical studies have shown that there is an important recognition site for the integrins in fibronectin⁴³ and collagen VI⁴⁴, a critical aspartate residue within a short peptide sequence (e.g. RGD, LDV). In collagen II, on the other hand, the role of such sequences in the interactions with integrins is not clear. As shown earlier²⁷, the linear peptides containing RGD sequences were able to inhibit cell adhesion to denatured collagen II, but failed to compete with the native collagen for the integrin mediated binding of cells. However, the cyclic peptides with RGD sequences inhibit the binding of α2β1 integrins to collagen⁴⁵, which indicates that the stable conformation of the peptide is critical for the functioning of an integrin recognition site. It was also shown that chondrocytes are able to migrate toward tetra-RGD containing peptides²². In human collagen II⁴⁶, one RGD and two RGD sequences per one a chain are located in the D3 and D4 period, respectively (see FIG. 9). Uniform binding of chondrocytes to all analyzed collagen II variants suggests, however, that the RGD-dependent mechanism is not significant for the β1 integrin mediated adhesion of chondrocytes to collagen II. Recently, Knight et al.²⁸ reported that in collagen I the GFPGER sequence is as a critical recognition site for the α1β1 and α2β1 integrins. Still, platelets that bind to collagen III via α2 β1 integrins^{47,48} use a different mechanism of interaction, since collagen III does not contain a GFPGER sequence⁴⁹. Attachment of chondrocytes to collagen II with a deleted D3 period, the only region of human collagen II that contains the GFPGER sequence, was not different in comparison to other collagen II variants. Presumably, other amino acid sequences, randomly distributed through the collagen II molecule, are able to support β1 mediated chondrocyte adhesion.

Migration of chondrocytes depends on the interactions of integrins with components of the extracellular matrix²². Clustering of integrins^{50, 51} and the density of extracellular ligands⁵² are important factors regulating cell migration. Data presented in this study demonstrate that collagen II supports the motility of chondrocytes. In the experiments with microtiter plates coated with different collagen II variants, chondrocytes were able to spread on full-length collagen II, and the collagen variants lacking the D1, D2 or D3 periods. However, the spreading was significantly altered when cells were cultured on the collagen II with deleted D4 period. The key role of the D4 period in the β1 integrin mediated migration of chondrocytes was also demonstrated in the experiments with the three-dimensional matrices. We have demonstrated that chondrocytes are not able to migrate into nanofibrous scaffolds neither when the D4 period is deleted from collagen II monomer, nor when the β1 integrin is selectively inactivated by antibodies. Our results do not provide an answer as to why the D4 period is critical for the chondrocyte spreading and migration on collagen II, and further studies will be required to find a minimal amino acid sequence of the D4 region that is critical for β1 dependent cell motility. As previously indicated, the D4 period contains two out of three RGD sequences present in human collagen II, and such clustering of the RGD sequences is critical for the migration of cells. As recently shown by Maheshwari et al.⁵³, the clustering of the YGRGD peptide immobilized on a synthetic polymer was able to reduce the average ligand density required to support cell migration. The D-staggered axial alignment of collagen monomers, and the presence of RGD sequences in the narrow region of a molecule arranges these sequences into clusters that form a well-defined pattern (FIG. 9). Such a pattern makes the surface of collagen fibril competent for the integrin-mediated migration of cells.

The results presented here indicate that collagen II consists of domains that differ in their ability to support attachment and migration of chondrocytes. Defining these sites is important for designing advanced collagen-based materials with multiple critical domains (see FIG. 10). Such a high density of these domains will enhance the ability of scaffolding material to support cells and, as a result, will promote tissue regeneration. In addition, the cassette system is suitable to characterize other sites of interactions, and this information can be used to engineer novel materials. For example, characterization of sites critical for interaction with bone morphogenetic proteins or collagenolytic enzymes will allow for the invention of collagen-based materials with improved characteristics important for delivery of growth factors and integrity of scaffolds.

### EXAMPLE 2

Engineering of DNA constructs encoding multi-D collagen II cassettes—To engineer DNA constructs encoding collagen-like proteins with multiplied particular D periods, the existing DNA cassettes corresponding to various regions of procollagen II were employed⁵⁴. The DNA cassettes set forth in Table 1 below were used.

TABLE 1
The regions of procollagen II encoded by the individual cassettes.

| | | | Restriction sites used | | |
|---|---|---|---|---|---|
| | | | in assembly of the | | |
| Cas- | Protein Domain | Amino acids | multi-D constructs | | |
| sette | Encoded | Encoded^{a} | A | B | C |
| Nt | N-propeptide and | 1-137 | Spel | — | Pvul |
| | N-telopeptide | | | | |
| D1 | D1-period of triple | 138-371 | SpeI | SrfI | BsrBI |
| | helix | | | | |
| D2 | D2-period of triple | 372-605 | SpeI | SrfI | BsrBI |
| | helix | | | | |
| D3 | D3-period of triple | 606-839 | SpeI | SrfI | |
| | helix | | | | |
| D4 | D4-period of triple | 840-1073 | SpeI | SrfI | BstuI |
| | helix | | | | |
| D5 | D5-period of triple | 1074-1151 | SpeI | SrfI | BsrBI |
| | helix | | | | |
| Ct | C-propeptide and | | SpeI | SrfI | — |
| | C-telopeptide | | | | |

^{a}Amino acids are numbered from methionine encoded by start codon.

The DNA cassettes were cloned into pcDNA2.1 vector (Invitrogen). To assemble DNA construct encoding multi-D4 collagen-like protein, the protocol described by Arnold et al.⁵⁴ for assembly of the DNA construct encoding normal procollagen II was used. To engineer the multi-D4 collagen-like protein, the following cloning steps were taken:

1. Ct+D5

2. CtD5+D4

3. CtD5D4+D4

4. CtD5D4D4+D4

5. CtD5D4D4D4+D1

6. CtD5D4D4D4D1+Nt

7. Final construct: CtD5D4D4D4D1Nt

The final construct (CtD5D4D4D4D1Nt, see FIG. 11) was cloned into mammalian expression vector (pcDNA3.1; Invitrogen). Using the same cloning strategy, DNA construct encoding the multi-D3 collagen-like protein was also engineered.

Expression of the multi-D cassettes DNA constructs—To express multi-D cassettes, the DNA constructs cloned into pcDNA3.1 vector were stably transfected into HT-1080 cells by calcium phosphate precipitation, and the G418-resistant clones were selected (see⁵⁵). The selected clones that secreted multi-D4 or multi-D3 collagen-like proteins were cultured under standard conditions without G418. To harvest the recombinant collagen-like proteins, the cells were cultured in Dulbecco's modified Eagle's medium supplemented with L-ascorbic acid phosphate magnesium salt n-hydrate (Wako; Osaka, Japan).

Purification of recombinant collagen-like proteins—Recombinant proteins were purified from culture media according to the method described by Fertala et al.⁵⁵. In brief, for each cell line, approximately 4 L of medium harvested from each 24-hr period was filtered through a 1.6 μm glass-fiber filter (Millipore) and supplemented with the following reagents at indicated concentrations: 0.1 M Tris-HCl buffer, 0.4 M NaCl, 25 mM EDTA, 10 mM N-ethylmaleimide, 1 mM p-aminobenzamidine, and 0.02% NaN₃ adjusted to pH 7.4. High molecular weight proteins in the medium were concentrated approximately 10-fold at 4° C. by the use of cartridges with a 100-kDa molecular weight cut-off (Prep/Scale-TFF filter; Millipore). Proteins in the concentrated media were precipitated overnight at 4° C. with 175 mg/ml of ammonium sulfate and collected by centrifugation at 15,000×g for 1 hr at 4° C. Procollagen II was purified using three-step ion exchange chromatography as described by Fertala et al.⁵⁵. Protein peak fractions were pooled and dialyzed against a storage buffer (0.1 M Tris-HCl buffer, pH 7.4, with 0.4 M NaCl and 10 mM EDTA). Finally, the purified collagen-like proteins were concentrated by ultrafiltration on a membrane filter (YM-100; Amicon) and stored at −80° C.

Analysis of thermal stability of novel collagen-like proteins. To determine whether novel collagen-like proteins were correctly folded and whether they were stable at physiological range of temperatures, the limited protease digestion assay was employed. Proteins were incubated in a programmable heating block. After reaching set temperature, the samples were incubated for additional 5 min. After that time mixture of trypsin (0.1 mg/ml) and chymotrypsin (0.25 mg/ml) was added to the samples for 2 min. followed by adding of electrophoresis running buffer and boiling. Overall, trypsin-chymotrypsin digestion was carried out at temperatures ranging from 25° C. to 42° C. Subsequently, products of digestion were separated in 7.5% polyacrylamide gels. The separated proteins were visualized by staining with Coomassie Blue (FIG. 12).

Cleavage of procollagen II with procollagen N- and C-proteinases—To analyze structural integrity of the propeptides of novel proteins, procollagen propeptides were enzymatically removed by cleavage with procollagen N-proteinase (EC 3.4.24.14) and procollagen C-proteinase (EC 3.4.24.19) purified from chick embryo tendons^{56, 57}. Enzymatic digestion was carried out in 25 mM Tris-HCl buffer, pH 7.5 containing 7 mM CaCl_{2, 100} mM NaCl, 0.015% Brij, and 0.02% NaN₃. The reaction mixture contained approximately 2 μg of procollagen, 1 units of N-proteinase, and 1 units of C-proteinase. One unit of each of these enzymes is defined as the amount of enzyme needed to cleave 1 μg of substrate in 1 h at 35° C. The reaction was carried out at 37° C. for 4 h. The enzymes were then inactivated by an addition of EDTA to a final concentration of 10 mM. Subsequently, products of enzymatic digestions were separated in 7.5% polyacrylamide gels. The separated proteins were visualized by staining with Coomassie Blue (FIGS. 13 and 14).

It will be apparent to those skilled in the art that various modifications and variations can be made to the present invention without departing from the spirit and scope of the invention. Thus, it is intended that the present invention cover the modifications and variations of this invention provided they come within the scope of the appended claims and their equivalents.

### BRIEF DESCRIPTION OF THE DRAWINGS

The accompanying drawings, which are incorporated in and constitute a part of this specification, illustrate embodiments of the invention and, together with the description, serve to explain the objects, advantages, and principles of the invention.

FIGS. 1A-B illustrate recombinant collagen II variants lacking particular D-periods. FIG. 1A is an electron microscopy of rotary shadowed recombinant procollagen II monomer. Segments of the molecule that correspond to particular regions, defined here as D-periods, are indicated by white bars. FIG. 1B is a polyacrylamide gel electrophoresis of the U chains of recombinant variants of collagen II. Recombinant collagen a chains with deletions of a complete D period migrate more rapidly than full-length a: chains but differently from each other because of variations in post-translational modifications^{29, 39}.

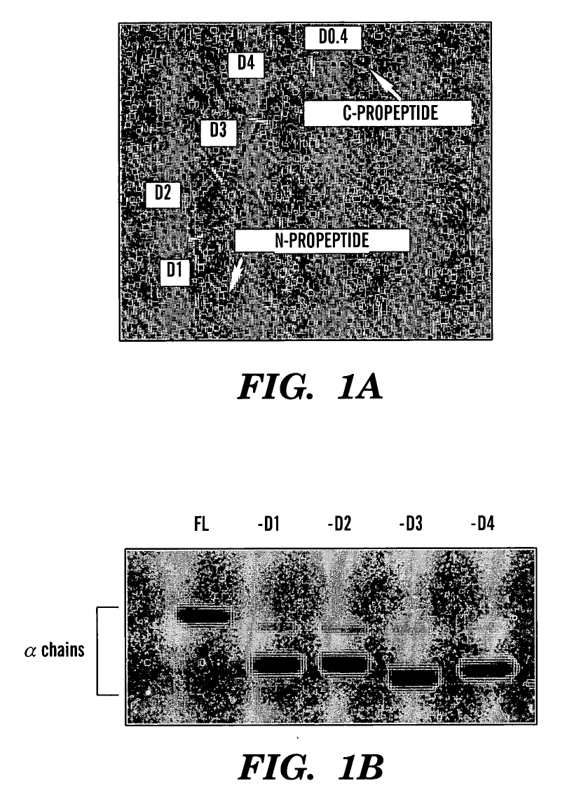

FIG. 2 is a schematic of the formation of nanofibrous matrices in the process of electrospinning.

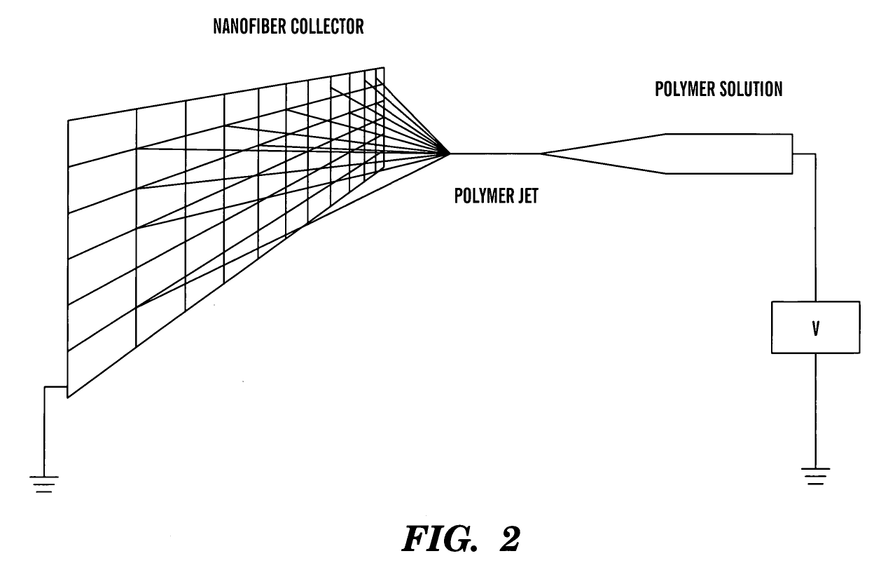

FIG. 3 shows attachment of human chondrocytes to the immobilized collagen II variants. Symbols: black bars—illustrate attachment of chondrocytes in the absence of anti-human β1 integrin antibodies; bars with a pattern illustrate attachment of chondrocytes in the presence of anti-human β1 integrin antibodies; F stands for plates coated with full-length collagen II, -D1, -D2 etc., plates coated with collagen II lacking specific D-periods; BSA stands for plates coated with bovine serum albumin.

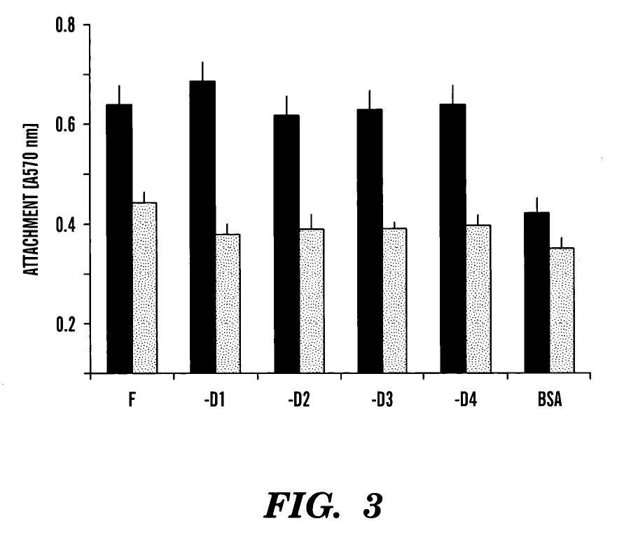

FIG. 4C shows chondrocytes grown on the plate coated with full-length collagen II (F). FIG. 4D shows chondrocytes grown on the plate coated with bovine serum albumin (BSA). FIG. 4A is a graphic representation of the surface area of cells grown on the collagen II variants and BSA. FIG. 4B shows spreading of chondrocytes cultured on the collagen II variants and BSA. In some experiments β1 integrin-mediated interactions were blocked with specific antibodies. The results are expressed as a percent of cells with the surface area equal to (±S.D.) or greater then the mean value of surface area of the cells grown on triple helical full-length collagen. Black bars represent cells seeded onto collagen variants, and white bars represent cells seeded onto triple helical collagen in the presence of anti-β1 integrin antibodies.

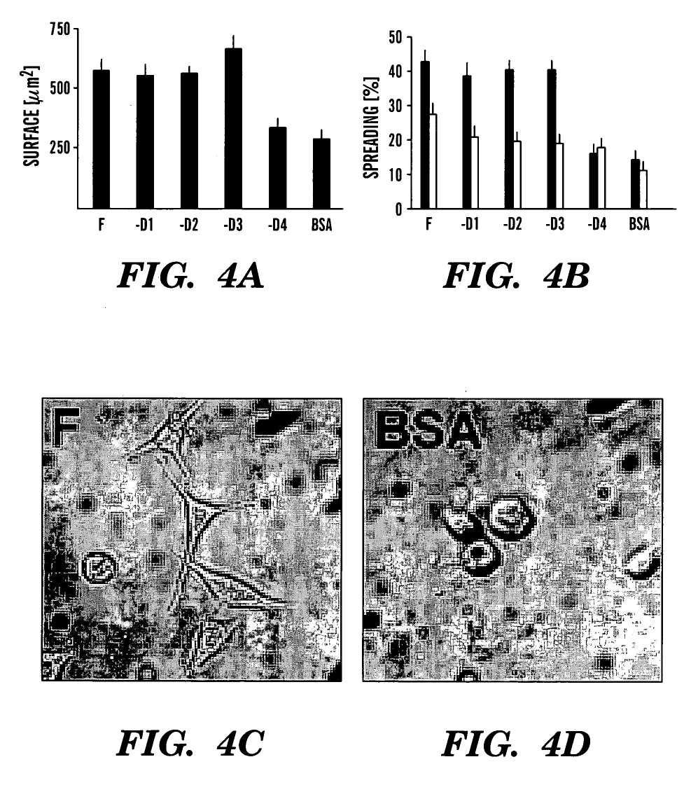

FIGS. 5A-H show growth of human fetal chondrocytes in nanofibrous matrices coated with recombinant collagen II variants with specifically deleted D-periods. FIGS. 5A-D are in 500× magnification; FIGS. 5E-H are in 1,500× magnification. FIGS. I-J show cells grown on full-length collagen II and -D4-coated nanofibrils; a view at a 14° angle; 1,500× magnification. Symbols: F stands for matrices coated with full-length collagen II, -D3, -D4-matrices coated with collagen II lacking D3 or D4 periods, BSA—matrices coated with bovine serum albumin. Bars: 10 μm.

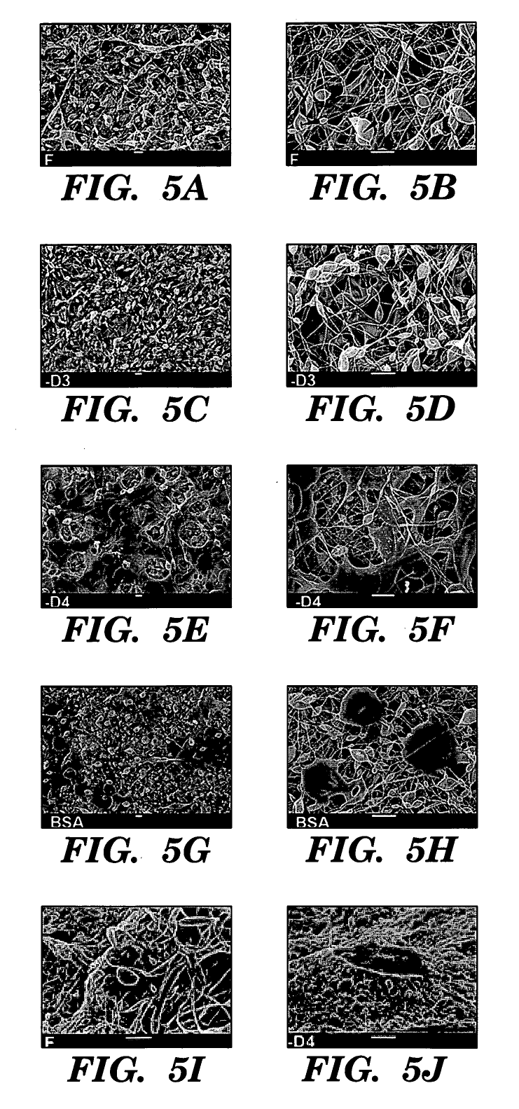

FIGS. 6A-B show growth of human fetal chondrocytes in nanofibrous matrices coated with recombinant full-length collagen II in the presence of anti β1 integrin antibodies. Note: in the presence of anti-β 1 integrin antibody cells do not migrate onto the scaffold.

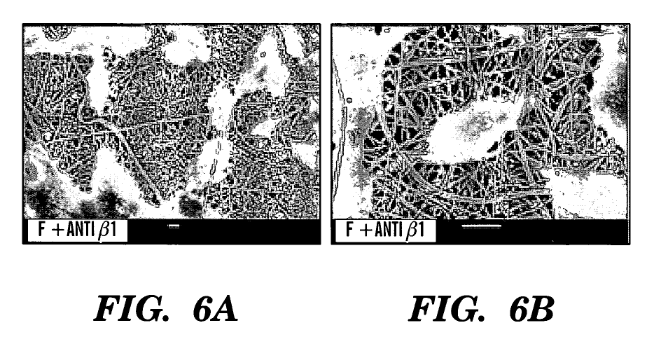

FIGS. 7A-B show Western blot analysis of collagen II and collagen 1X synthesized by chondrocytes grown on nanofibrillar matrix coated with full-length collagen II after 50 days of culture. FIG. 7A shows proteins immunostained with the anti-collagen II antibodies. FIG. 7B shows proteins immunostained with the anti-collagen IX antibodies. Symbols: M_{II}, M_{IX} collagen II and collagen IX markers.

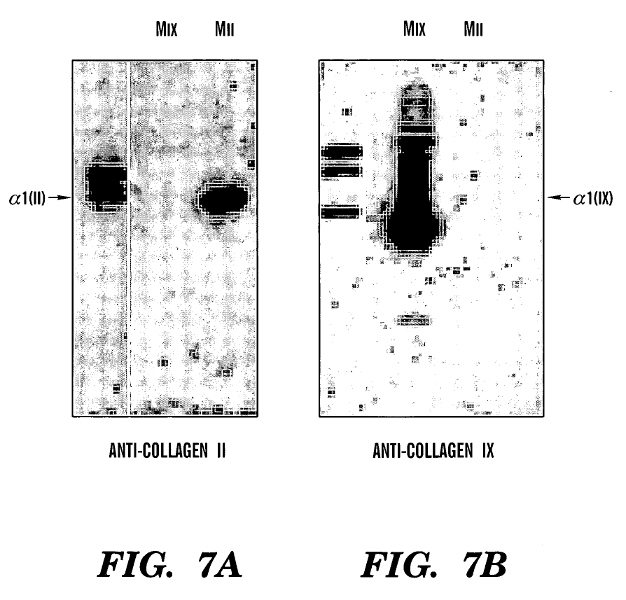

FIG. 8 shows an electron microscopy analysis of matrix assembled by chondrocytes cultured for 50 days on a surface of nanofibrillar scaffold coated with full-length recombinant collagen II. Arrows indicate collagen II fibrils deposited between chondrocytes. Insert: Detail showing collagen fibrils with apparent periodicity. Symbols: CH; chondrocyte, CF: collagen fibrils. Bar: 100 nm.

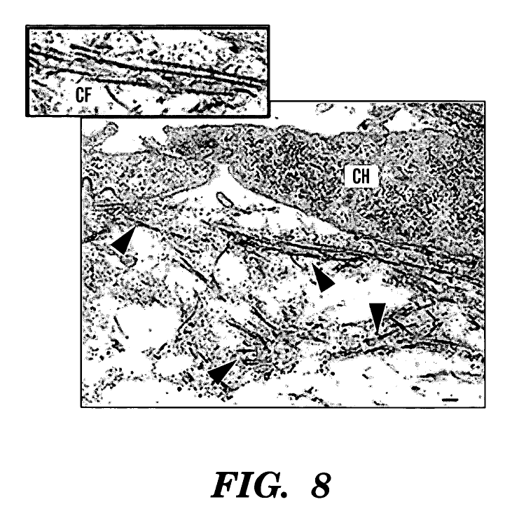

FIG. 9 is a schematic of the D-periodic organization of monomers in collagen fibril. Sections of the monomers represent collagen D-periods. Thick lines indicate RGD sequences.

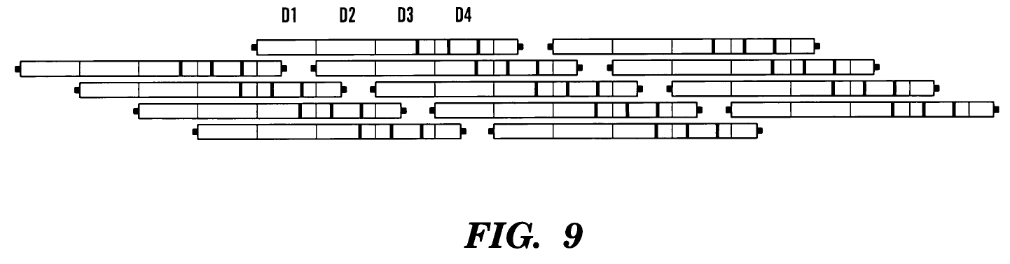

FIG. 10 illustrates the use of collagen cassette system for mapping critical interaction sites. Collagen II variants lacking specific D-periods are used to map sites important for interaction with enzymes, growth factors, and cells. The schematic illustrates the collagen II fragments that are critical for supporting chondrocytes. Consequently, the “super collagen” containing multiplied interaction sites will be used to prepare a scaffold with novel biological characteristics.

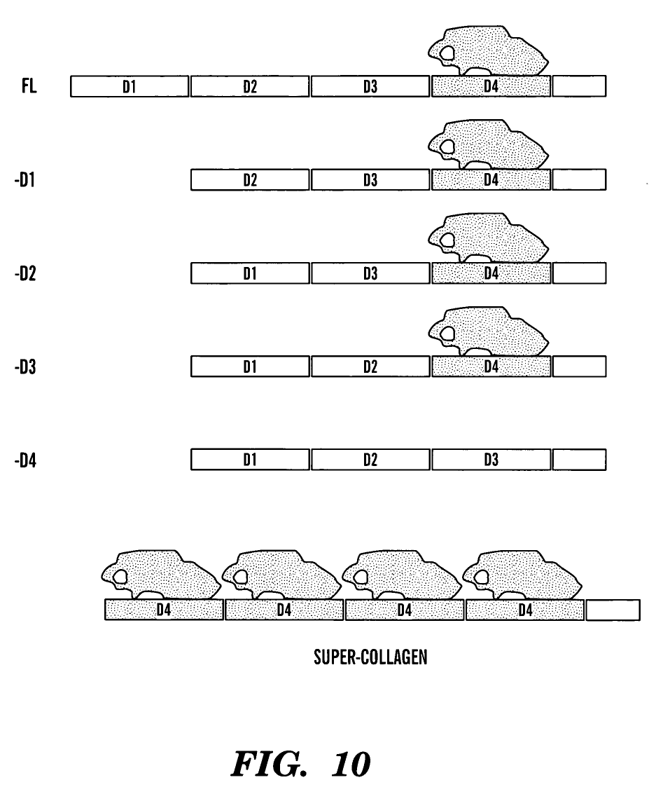

FIG. 11 shows an assembly of a DNA construct encoding multi D4 collagen-like protein. The DNA fragments constructed during each step of assembly of the multi-D4 DNA construct are indicated.

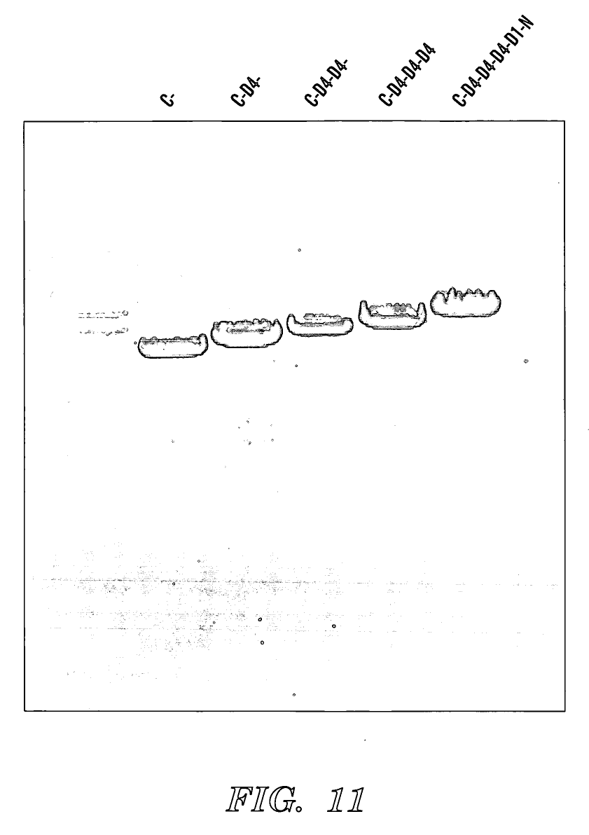

FIG. 12 is an analysis of structural integrity of novel collagen like protein. NOTE: multi D4-collagen-like protein is stable up to 42° C., which indicate correct folding of triple helical structure.

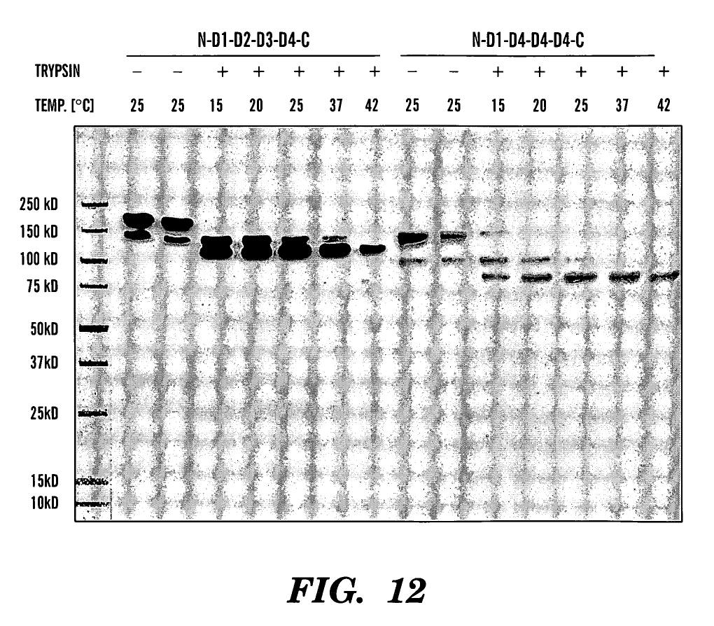

FIG. 13 shows cleavage of recombinant multi D4 procollagen-like protein (mD4) with procollagen N-proteinase. NOTE: correct processing of the N-propeptide is an indicative of correctly folded N-propeptide. Symbols: pro-II; normal procollagen II, pro-mD4; multi D4 procollagen, pC-TI; product derived from cleavage of procollagen with procollagen N-proteinase, pC-mD4; product derived from cleavage of the pro-mD4 with procollagen N-proteinase. Apparent difference in mass of procollagen II and pro-mD4 is most likely due to differences in posttranslational modifications between two proteins.

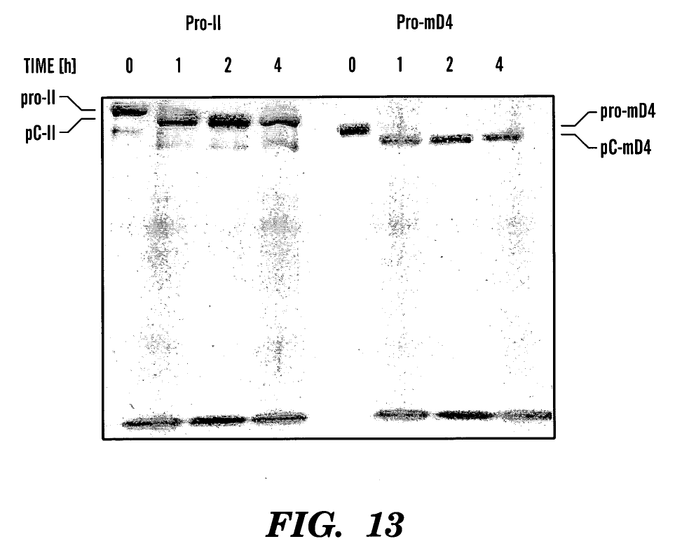

FIG. 14 shows cleavage of recombinant multi D4 procollagen-like protein (mD4) with procollagen C-proteinase. NOTE: correct processing of the C-propeptide is an indicative of correctly folded C-propeptide. Symbols: pro-II; normal procollagen II, pro-mD4; multi D4 procollagen, pN-II; product derived from cleavage of procollagen with procollagen C-proteinase, pN-mD4; product derived from cleavage of the pro-mD4 with procollagen C-proteinase. Apparent difference in mass of procollagen II and pro-mD4 is most likely due to differences in post-translational modifications between two proteins.

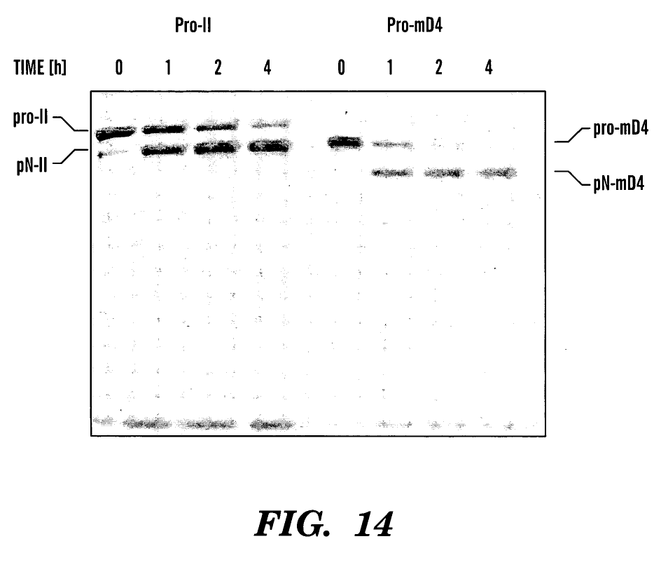

### REFERNCES

The references cited below and incorporated throughout the application are incorporated herein by reference.

1. Vunjak-Novakovic G, Obradovic B, Martin I, Bursac P M, Langer R, Freed L E. Dynamic cell seeding of polymer scaffolds for cartilage tissue engineering. Biotechnol Prog 1998; 14:193-202.

2. Sams A E, Minor R R, Wootton J A, Mohammed H, Nixon A J. Local and remote matrix responses to chondrocyte-laden collagen scaffold implantation in extensive articular cartilage defects. Osteoarthritis Cartilage 1995; 3:61-70.

3. Peretti G M, Randolph M A, Caruso E M, Rossetti F, Zaleske D J. Bonding of cartilage matrices with cultured chondrocytes: an experimental model. J Orthop Res 1998; 16:89-95.

4. Martin I, Padera R F, Vunjak-Novakovic G, Freed L E. In vitro differentiation of chick embryo bone marrow stromal cells into cartilaginous and bone-like tissues. J Orthop Res 1998; 16:181-9.

5. Gillogly S D, Voight M, Blackburn T. Treatment of articular cartilage defects of the knee with autologous chondrocyte implantation. J Orthop Sports Phys Ther 1998; 28:241-51.

6. Breitbart A S, Grande D A, Kessler R, Ryaby J T, Fitzsimmons R J, Grant R T. Tissue engineered bone repair of calvarial defects using cultured periosteal cells. Plast Reconstr Surg 1998; 101:567-74; discussion 575-6.

7. Aigner J, Tegeler J, Hutzler P, Campoccia D, Pavesio A, Hammer C, Kastenbauer E, Naumann A. Cartilage tissue engineering with novel nonwoven structured biomaterial based on hyaluronic acid benzyl ester. J Biomed Mater Res 1998; 42:172-81.

8. Cao Y, Rodriguez A, Vacanti M, Ibarra C, Arevalo C, Vacanti C A. Comparative study of the use of poly(glycolic acid), calcium alginate and pluronics in the engineering of autologous porcine cartilage. J Biomater Sci Polym Ed 1998; 9:475-87.

9. Nehrer S, Breinan H A, Ramappa A, Shortkroff S, Young G, Minas T, Sledge C B, Yannas I V, Spector M. Canine chondrocytes seeded in type I and type II collagen implants investigated in vitro [published erratum appears in J Biomed Mater Res 1997 Winter;38(4):288]. J Biomed Mater Res 1997; 38:95-104.

10. Reddi A H. Bone and cartilage differentiation. Curr Opin Genet Dev 1994; 4:737-44.

11. Prockop D J, Kivirikko K I. Collagens: molecular biology, diseases, and potentials for therapy. Annu Rev Biochem 1995; 64:403-34.

12. Wu J J, Eyre D R, Slayter H S. Type V I collagen of the intervertebral disc. Biochemical and electron-microscopic characterization of the native protein. Biochem J 1987; 248:373-81.

13. Li S W, Prockop D J, Helminen H, Fassler R, Lapvetelainen T, Kiraly K, Peltarri A, Arokoski J, Lui H, Arita M, et al. Transgenic mice with targeted inactivation of the Col2 alpha 1 gene for collagen II develop a skeleton with membranous and periosteal bone but no endochondral bone. Genes Dev 1995; 9:2821-30.

14. Yang C, Li S W, Helminen H J, Khillan J S, Bao Y, Prockop D J. Apoptosis of chondrocytes in transgenic mice lacking collagen II. Exp Cell Res 1997; 235:370-3.

15. Dziadek M, Darling P, Bakker M, Overall M, Zhang R Z, Pan T C, Tillet E, Timpl R, Chu M L. Deposition of collagen VI in the extracellular matrix during mouse embryogenesis correlates with expression of the alpha 3(VI) subunit gene. Exp Cell Res 1996; 226:302-15.

16. Makihira S, Yan W, Ohno S, Kawamoto T, Fujimoto K, Okimura A, Yoshida E, Noshiro M, Hamada T, Kato Y. Enhancement of cell adhesion and spreading by a cartilage-specific noncollagenous protein, cartilage matrix protein (CMP/Matrilin-1), via integrin alpha1beta1. J Biol Chem 1999; 274:11417-23.

17. Loeser R F, Sadiev S, Tan L, Goldring M B. Integrin expression by primary and immortalized human chondrocytes: evidence of a differential role for alpha1beta1 and alpha2beta1 integrins in mediating chondrocyte adhesion to types II and VI collagen [In Process Citation]. Osteoarthritis Cartilage 2000; 8:96-105.

18. Buckwalter J A, Mankin H J. Articular cartilage: tissue design and chondrocyte-matrix interactions. Instr Course Lect 1998; 47:477-86.

19. Svoboda K K. Chondrocyte-matrix attachment complexes mediate survival and differentiation. Microsc Res Tech 1998; 43:111-22.

20. Loeser R F. Integrin-mediated attachment of articular chondrocytes to extracellular matrix proteins. Arthritis Rheum 1993; 36:1103-10.

21. Shakibaei M, De Souza P, Merker H J. Integrin expression and collagen type II implicated in maintenance of chondrocyte shape in monolayer culture: an immunomorphological study. Cell Biol Int 1997; 21:115-25.

22. Shimizu M, Minakuchi K, Kaji S, Koga J. Chondrocyte migration to fibronectin, type I collagen, and type II collagen. Cell Struct Funct 1997; 22:309-15.

23. Camper L, Hellman U, Lundgren-Akerlund E. Isolation, cloning, and sequence analysis of the integrin subunit alpha10, a beta1-associated collagen binding integrin expressed on chondrocytes. J Biol Chem 1998; 273:20383-9.

24. von der Mark K, Mollenhauer J. Annexin V interactions with collagen. Cell Mol Life Sci 1997; 53:539-45.

25. Mollenhauer J, Mok M T, King K B, Gupta M, Chubinskaya S, Koepp H. Cole A A. Expression of anchorin CII (cartilage annexin V) in human young, normal adult, and osteoarthritic cartilage. J Histochem Cytochem 1999; 47:209-20.

26. Woods V L, Jr., Schreck P J, Gesink D S, Pacheco H O, Amiel D, Akeson W H, Lotz M. Integrin expression by human articular chondrocytes. Arthritis Rheum 1994; 37:537-44.

27. Tuckwell D S, Ayad S, Grant M E, Takigawa M, Humphries M J. Conformation dependence of integrin-type II collagen binding. Inability of collagen peptides to support alpha 2 beta 1 binding, and mediation of adhesion to denatured collagen by a novel alpha 5 beta 1-fibronectin bridge. J Cell Sci 1994; 107:993-1005.

28. Knight C G, Morton L F, Peachey A R, Tuckwell D S, Farndale R W, Barnes M J. The collagen-binding A-domains of integrins alpha(1)beta(1) and alpha(2)beta(1) recognize the same specific amino acid sequence, GFOGER, in native (triple-helical) collagens. J Biol Chem 2000; 275:35-40.

29. Arnold W V, Sieron A L, Fertala A, Bachinger H P, Mechling D, Prockop D J. A cDNA cassette system for the synthesis of recombinant procollagens. Variants of procollagen II lacking a D-period are secreted as triple-helical monomers. Matrix Biol 1997; 16:105-16.

30. Fertala A, Sieron A L, Ganguly A, Li S W, Ala-Kokko L, Anumula K R, Prockop D J. Synthesis of recombinant human procollagen II in a stably transfected tumour cell line (HT1080). Biochem J 1994; 298:31-7.

31. Myllyharju J. Recombinant collagen trimers from insect cells and yeast [In Process Citation]. Methods Mol Biol 2000; 139:39-48.

32. Toman P D, Pieper F, Sakai N, Karatzas C, Platenburg E, de Wit I, Samuel C, Dekker A, Daniels G A, Berg R A, Platenburg G J. Production of recombinant human type I procollagen homotrimer in the mammary gland of transgenic mice. Transgenic Res 1999; 8:415-27.

33. Ruggiero F, Exposito J Y, Bournat P, Gruber V, Perret S, Comte J, Olagnier B, Garrone R, Theisen M. Triple helix assembly and processing of human collagen produced in transgenic tobacco plants. FEBS Lett 2000; 469:132-6.

34. Piez K A. Extracellular Matrix Biochemistry. In: Piez K A, and Reddi, A. H., ed. New York: Elsevier, 1984:1-40.

35. Reginato A M, Iozzo R V, Jimenez S A. Formation of nodular structures resembling mature articular cartilage in long-term primary cultures of human fetal epiphyseal chondrocytes on a hydrogel substrate. Arthritis Rheum 1994; 37:1338-49.

36. Zheng J, Northrup S R, Hornsby P J. Modification of materials formed from poly(L-lactic acid) to enable covalent binding of biopolymers: application to high-density three-dimensional cell culture in foams with attached collagen. In Vitro Cell Dev Biol Anim 1998; 34:679-84.

37. Norris I D, Shaker M S, Ko F K, MacDiarmid A G. Electrostatic fabrication of ultrafine conducting fibers: polyaniline/polyethylene oxide blends. Synthetic Metals 2000; 114:109-114.

38. Woessner J F. The determination of hydroxyprolinin tisue and protein samples containing small proportions of this imino acid. ArchivBiochem.Biophys. 1961; 93:440-447.

39. Arnold W V, Fertala A, Sieron A L, Hattori H, Mechling D, Bachinger H P, Prockop D J. Recombinant procollagen II: Deletion of D period segments identifies sequences that are required for helix stabilization and generates a temperature-sensitive N-proteinase cleavage site. J Biol Chem 1998; 273:31822-8.

40. Fong H, Chun I, Reneker D H. Beaded Nanofibers Formed During Electrospinning. Polymer 1999; 40:4585-4592.

41. Enomoto M, Leboy P S, Menko A S, Boettiger D. Beta 1 integrins mediate chondrocyte interaction with type I collagen, type II collagen, and fibronectin. Exp Cell Res 1993; 205:276-85.

42. Durr J, Goodman S, Potocnik A, von der Mark H, von der Mark K. Localization of beta 1-integrins in human cartilage and their role in chondrocyte adhesion to collagen and fibronectin. Exp Cell Res 1993; 207:235-44.

43. Buck C A, Horwitz A F. Cell surface receptors for extracellular matrix molecules. Annu Rev Cell Biol 1987; 3:179-205.

44. Marcelino J, McDevitt C A. Attachment of articular cartilage chondrocytes to the tissue form of type VI collagen. Biochim Biophys Acta 1995; 1249:180-8.

45. Cardarelli P M, Yamagata S, Taguchi I, Gorcsan F, Chiang S L, Lobl T. The collagen receptor alpha 2 beta 1, from MG-63 and HT1080 cells, interacts with a cyclic RGD peptide. J Biol Chem 1992; 267:23159-64.

46. Su M W, Lee B, Ramirez F, Machado M, Horton W. Nucleotide sequence of the full length cDNA encoding for human type II procollagen. Nucleic Acids Res 1989; 17:9473.

47. Staatz W D, Walsh J J, Pexton T, Santoro S A. The alpha 2 beta 1 integrin cell surface collagen receptor binds to the alpha 1 (I)-CB3 peptide of collagen. J Biol Chem 1990; 265:4778-81.

48. Sixma J J, Hindriks G, Van Breugel H, Hantgan R, de Groot P G. Vessel wall proteins adhesive for platelets. J Biomater Sci Polym Ed 1991; 3:17-26.

49. Ala-Kokko L, Kontusaari S, Baldwin C T, Kuivaniemi H, Prockop D J. Structure of cDNA clones coding for the entire prepro alpha 1 (III) chain of human type III procollagen. Differences in protein structure from type I procollagen and conservation of codon preferences. Biochem J 1989; 260:509-16.

50. Dedhar S, Hannigan G E. Integrin cytoplasmic interactions and bidirectional transmembrane signalling. Curr Opin Cell Biol 1996; 8:657-69.

51. Hato T, Pampori N, Shattil S J. Complementary roles for receptor clustering and conformational change in the adhesive and signaling functions of integrin alpha1Ib beta3. J Cell Biol 1998; 141:1685-95.

52. Kuntz R M, Saltzman W M. Neutrophil motility in extracellular matrix gels: mesh size and adhesion affect speed of migration. Biophys J 1997; 72:1472-80.

53. Maheshwari G, Brown G, Lauffenburger D A, Wells A, Griffith L G. Cell adhesion and motility depend on nanoscale RGD clustering. J Cell Sci 2000; 113:1677-1686.

54. Arnold W V, Sieron A L, Fertala A, Bachinger H P, Mechling D, Prockop D J. A cDNA cassette system for the synthesis of recombinant procollagens. Variants of procollagen II lacking a D-period are secreted as triple-helical monomers. Matrix Biol.1997; 16(3): 105-16.

55. Fertala A, Sieron A L, Ganguly A, et al. Synthesis of recombinant human procollagen II in a stably transfected tumor cell line (HT1080). Biochem J.1994; 298(Pt 1): 31-7.

56. Hojima Y, McKenzie J A, van der Rest M, Prockop D J. Type I procollagen N-proteinase from chick embryo tendons. Purification of a new 500-kDa form of the enzyme and identification of the catalytically active polypeptides. J Biol Chem.1989; 264(19): 11336-45.

57. Hojima Y, van der Rest M, Prockop D J. Type I procollagen carboxyl-terminal proteinase from chick embryo tendons. Purification and characterization. J Biol Chem.1985; 260(29): 15996-6003.

1. A recombinant collagen-like protein comprising a multi-domain of the formula (D-D)x, wherein D is selected from D1, D2, D3, D4 or D5 protein domain of collagen and each D is identical, and wherein x is 1-5.

2. The recombinant collagen-like protein of claim 1, wherein x is 2.

3. A recombinant collagen-like protein having the structure CtD5D4D4D4D1Nt.

4. A nucleic acid sequence, encoding the recombinant collagen-like protein of claims 1-3.

5. A host cell comprising the nucleic acid of claim 4.

6. The host cell of claim 5, wherein the cell is a prokaryotic cell.
